(19) United States
(12) Patent Application Publication
(10) Pub. No.: US 2015/0041711 A1
(43) Pub. Date: Feb. 12, 2015

(54) LOW VISCOSITY DIAROYL PASTE AND METHOD FOR MAKING THE SAME

(71) Applicant: United Initiators SPI, Inc., Helena, AR (US)

(72) Inventors: Kevin S. Guigley, Hernando, MS (US); Brian S. Helton, Helena, AR (US)

(21) Appl. No.: 14/452,972

(22) Filed: Aug. 6, 2014

Related U.S. Application Data
(60) Provisional application No. 61/862,751, filed on Aug. 6, 2013.

(51) Int. Cl. C07C 409/32; C07C 407/00

(52) U.S. Cl. CPC C07C 409/32; C07C 407/006
USPC 252/186.26

(57) ABSTRACT

In various embodiments, the present invention pertains to diaroyl peroxide compositions and methods for making the same.

### CROSS-REFERENCE TO RELATED APPLICATIONS

This application claims priority to and the benefit of, and incorporates herein by reference in its entirety, U.S. Provisional Patent Application No. 61/862,751, entitled LOW VISCOSITY DIAROYL PEROXIDE PASTE AND PROCESS FOR MAKING THE SAME, which was filed on Aug. 6, 2013.

### FIELD OF THE INVENTION

In various embodiments, the present disclosure pertains to diaroyl peroxide compositions and methods for making the same.

### BACKGROUND

Diaroyl peroxides, and dibenzoyl peroxide in particular, are commonly used as initiators for polymerization reactions in the preparation of vinyl polymers, curing agents for unsaturated resins, and as bleaching agents, as well as for acne treatment, dyeing hair, flour preparation, and other uses. One particular use for diaroyl peroxides is in the production of polymer concrete boxes. These boxes are typically used to protect outdoor electrical switches, electrical connectors, and water valves. The box is made by molding a mixture of polymer resin, fillers, and a peroxide initiator. In certain cases, dibenzoyl peroxide is used as the initiator. The initiator starts the chemical reaction that results in the polymer resin transforming from a liquid to a solid material. This process results in a plastic container that is weather resistant and an electrical insulator.

Another particular use for diaroyl peroxides as polymerization initiators is in the securing of mine bolts. Mine bolts, also known as rock bolts, are used to anchor strata and safety devices in underground structures. A hole is bored in the ceiling or wall of a mine or other tunnel, and a cartridge containing a securing compound is inserted into the hole. The securing compound may be a mixture of diaroyl peroxide, limestone, polymer resin, and a thickening agent. A mine bolt, also known as a roof bolt, is inserted into the hole, puncturing the cartridge and releasing its contents. The contents of the cartridge solidify into a concrete-like material that fixes the mine bolt in place. This use of a securing compound aids in the prevention of the mine bolt becoming loosened or dislodged, which increases the level of safety within the underground structure.

Diaroyl peroxides compositions are often stored, transported, and used in the form of stabilized liquid dispersions in paste form. The viscosity of these pastes typically varies with the concentration of diaroyl peroxide, with higher concentrations resulting in higher viscosity. Diaroyl peroxide liquid dispersions are typically referred to as “pastes” in the industry regardless of their viscosity.

Most diaroyl peroxides, and dibenzoyl peroxide in particular, are hazardous in their dry crystalline form and require diluents, also referred to as phlegmatizers or desensitizers, to facilitate their safe handling. Diaroyl peroxides, when manufactured, are typically in the form of small granules which are agglomerates of very fine crystals. An important property in the selection of diluents for a diaroyl peroxide composition is that it softens the granules so they disperse or break down readily into individual crystals. This eliminates the necessity of milling the composition in order to reduce particle size. Thus a desirable characteristic of a suitable diluent is that it softens or disperses the granules readily.

Dibenzoyl peroxide is commonly referred to as benzoyl peroxide or BPO in the industry. BPO pastes comprising less than about 45% BPO by weight typically have a flowable consistency, such that they may be transported and stored as liquids. By lacking a flowable consistency, higher concentration BPO pastes require special handling for transportation and storage. For example, BPO pastes comprising between about 50% and 55% BPO by weight typically have a viscosity greater than about 100,000 cP at 20° C.

Diaroyl peroxide pastes may be stored in non-temperature controlled environments. In cold climates, there is a risk that diaroyl peroxide pastes may freeze. For example, pastes with a concentration of about 55% BPO produced using techniques common in the industry typically have a freezing point within the range of about −11° C. to about −13° C. Upon freezing, water separates from the liquid dispersion. Due to the high viscosity of compositions containing higher concentrations of diaroyl peroxide, it is very difficult to reconstitute the compositions after freezing. Accordingly, a need exists for a diaroyl peroxide composition with decreased viscosity and a lower freezing point.

### SUMMARY

Embodiments disclosed herein provide diaroyl peroxide compositions with decreased viscosity characteristics, improved freezing resistance and improved stability. The term stability, unless otherwise noted, refers to chemical stability, i.e., resistance to loss of activity over time or decomposition. Heat is evolved during the decomposition of diaroyl peroxides, and exposure to increased temperature correspondingly increases the rate of decomposition of diaroyl peroxides. Improved chemical stability widens the range of temperatures and conditions at which a diaroyl peroxide composition can be shipped, stored, or used.

Disclosed herein is a method of making a diaroyl peroxide paste, the method comprising blending a diaroyl peroxide, at least one surfactant, and at least one diluent, waiting a predetermined period of time, and adding paraffin oil, wherein the paste is at least about 45% by weight diaroyl peroxide and has a viscosity of less than about 10,000 cP. In some embodiments, the paste is not agitated during the waiting. In further embodiments, the predetermined period of time is at least 5 minutes, at least 15 minutes or at least 30 minutes. In some embodiments, the method further comprises adding a dispersant. In certain embodiments, the dispersant is one of water, ethylene glycol, propylene glycol, glycerin, propylene carbonate, and hexylene glycol. In further embodiments, the dispersant is water. In certain embodiments, the water is added subsequent to the paraffin oil. In some embodiments, the method further comprises adding a salt or a water soluble salt. In further embodiments, the salt is added subsequent to the addition of paraffin oil. In certain embodiments, the salt is one of sodium chloride, an alkali carboxylic acid, or mixture thereof. In some embodiments, the diaroyl peroxide is dibenzoyl peroxide. In further embodiments, the at least one diluent is one of a phthalate, benzoate and dibenzoate, phosphate, citrate, or adipate, or mixtures thereof. In certain embodiments, the at least one diluent is a benzyl ester or mixture of benzyl esters. In some embodiments, the paste is at least about 50% by weight diaroyl peroxide, and wherein the predetermined period of time is at least 15 minutes. In further embodiments, the paste is at least about 53% by weight diaroyl peroxide and has a viscosity of less than about 5,000 cP or less than about 1,000 cP. In certain embodiments, the paraffin oil is unsubstituted. In further embodiments, the paraffin oil is not halogenated. In some embodiments, the paraffin oil is not chlorinated. In further embodiments, the diaroyl peroxide is unsubstituted.

Disclosed herein is a diaroyl peroxide composition, the composition comprising at least about 45% by weight diaroyl peroxide, between about 2% and about 15% by weight diluent, between about 0.1% and about 10% by weight surfactant, and between about 1% and about 30% by weight paraffin oil, wherein the composition has a viscosity of less than about 10,000 cP and a freezing point of less than about −20° C. In some embodiments, the composition includes at least about 50% by weight diaroyl peroxide, between about 4% and about 10% by weight diluent, between about 0.5% and about 7% by weight surfactant, and between about 5% and about 25% by weight paraffin oil, and wherein the composition has a viscosity of less than about 5,000 cP. In further embodiments, the composition further comprises between about 5% and about 35% by weight dispersant. In certain embodiments, the dispersant is water. In some embodiments, the composition includes at least about 50% by weight diaroyl peroxide and has a viscosity of less than about 5,000 cP. In further embodiments, the composition has a viscosity of less than about 1,000 cP. In certain embodiments, the paraffin oil is unsubstituted. In further embodiments, the paraffin oil is not halogenated. In some embodiments, the paraffin oil is not chlorinated. In further embodiments, the diaroyl peroxide is unsubstituted.

Disclosed herein is a diaroyl peroxide composition, the composition comprising at least about 50% by weight diaroyl peroxide, between about 4% and about 10% by weight diluent, between about 6% and about 24% by weight paraffin oil, and between about 12% and about 30% by weight dispersant, wherein the composition has a viscosity of less than about 10,000 cP and a freezing point of less than about −20° C. In some embodiments, the composition further comprises between about 0.1% and about 5% by weight of a stabilizer or a preservative. In certain embodiments, the composition further comprises between about 0.1% and about 1% by weight of a stabilizer or a preservative. In further embodiments, the dispersant is water. In certain embodiments, the composition further comprises at least one of a carbonate, phosphate, sulfate or alkaline mineral. In some embodiments, the composition further comprises calcium carbonate or calcium sulfate. In further embodiments, the composition further comprises a polymer resin and a thickening agent. In certain embodiments, the paraffin oil is unsubstituted. In further embodiments, the paraffin oil is not halogenated. In some embodiments, the paraffin oil is not chlorinated. In further embodiments, the diaroyl peroxide is unsubstituted.

Disclosed herein is a diaroyl peroxide composition, the composition comprising at least about 50% by weight diaroyl peroxide, between about 4% and about 10% by weight diluent, between about 0.5% and about 7% by weight surfactant, between about 5% and about 25% by weight non-polar solvent with an octanol partition log p of at least about 5; and wherein the composition has a viscosity of less than about 5,000 cP. In some embodiments, the non-polar solvent is one of paraffin oil, naptha or kerosene.

This summary is provided to introduce a selection of the concepts that are described in further detail in the detailed description contained herein. This summary is not intended to identify any primary or essential features of the claimed subject matter. Each embodiment described herein is not intended to address every object described herein, and each embodiment does not necessarily include each feature described. Other forms, embodiments, objects, advantages, benefits, features, and aspects of the present invention will become apparent to one of skill in the art from the detailed description and drawings contained herein.

### DETAILED DESCRIPTION OF THE PREFERRED EMBODIMENT

Embodiments of the present invention include a multi-step process for producing a diaroyl peroxide composition with decreased viscosity characteristics and improved freezing resistance and improved stability. The first step comprises blending a diaroyl peroxide with at least one diluent and at least one surfactant. After waiting for a predetermined, non-zero period of time, paraffin oil is added. In some embodiments, a stabilizer is added to the paste. In certain embodiments, one or more preservatives are added to the paste. In some embodiments, organic peroxides are added to the paste. Example organic peroxides include cumene hydroperoxide and tert-butyl-peroxybenzoate. In some embodiments, a third step comprises adding water to the paste subsequent to the addition of the paraffin oil. In certain embodiments, at least one salt is added to the paste during the first step or with the water to decrease water separation.

The term paraffin oil, as used herein, refers to an alkane or, more commonly, a mixture of alkanes that adopt a liquid state at room temperature. Paraffin oil may include linear and branched alkanes, typically ranging in size from C5 to C20. In some embodiments, the diaroyl peroxide paste includes an amount of paraffin oil within the range of about 1% to about 30% by weight. In further embodiments, the diaroyl peroxide paste includes an amount of paraffin oil within the range of about 5% to about 25% by weight. In certain embodiments, the diaroyl peroxide paste includes an amount of paraffin oil within the range of about 8% to about 20% by weight.

Paraffin oil has been used in diaroyl peroxide pastes with diaroyl peroxide concentrations of less than about 10% by weight. However, paraffin oil unexpectedly provides a significant decrease in viscosity when added subsequently to blending the diaroyl peroxide, diluent and surfactant ingredients in higher concentration diaroyl peroxide compositions. This decrease in diaroyl peroxide paste viscosity does not occur when paraffin oil is simultaneously blended with the diaroyl peroxide and dispersing agent ingredients, which is the common practice in the industry.

Naptha is mixture of aromatic organic compounds, typically substituted and unsubstituted cycloalkanes, that adopt liquid or solid states at room temperature. Naptha and paraffin oil are both non-polar solvents with octanol/water partition coefficients (log P) of greater than about 5.

Diaroyl peroxides, such as BPO, can form small granules which are agglomerates of fine crystals. The term diluent, as used herein, refers to a substance that acts as a softening agent, softening the granules so that they disperse into the paste. Diluents, for example, phthalates, benzoates and dibenzoates, phosphates, citrates, adipates, and mixtures thereof, are known to be useful in dispersing diaroyl peroxides. In some embodiments, the at least one diluent is a benzyl ester, such as isodecyl benzyl ester, 2-ethylhexyl benzoate, dipropylene glycol dibenzoate, or diethylyene glycol dibenzoate, or combinations thereof. In some embodiments, the diaroyl peroxide paste includes an amount of diluent within the range of about 1% to about 20% by weight. In further embodiments, the diaroyl peroxide paste includes an amount of diluent within the range of about 2% to about 15% by weight. In some embodiments, the diaroyl peroxide paste includes an amount of diluent within the range of about 4% to about 10% by weight. In certain embodiments, the diaroyl peroxide paste includes an amount of diluent within the range of about 6% to about 8% by weight.

In some embodiments, the at least one surfactant is a non-ionic surfactant. In certain embodiments, the at least one surfactant is a mixture of two or more non-ionic surfactants. Examples of suitable surfactants include poly (ethylene oxide—co—propylene oxide) copolymers, polyglycerol esters of fatty acids, alkoxylated fatty alcohols, and benzyl poly ethylene oxide alkyl phenyl ethers. In some embodiments, the diaroyl peroxide paste includes an amount of at least one surfactant within the range of about 0.1% to about 10% by weight. In further embodiments, the diaroyl peroxide paste includes an amount of at least one surfactant within the range of about 0.1% to about 7% by weight. In certain embodiments, the diaroyl peroxide paste includes an amount of at least one surfactant within the range of about 0.1% to about 5% by weight. In some embodiments, the diaroyl peroxide paste includes an amount of at least one surfactant within the range of about 0.1% to about 3% by weight.

The term dispersant, as used herein, refers to a substance added to a suspension to improve the separation of particles and to prevent settling or clumping. Water has been used as dispersant for many organic peroxides including diaroyl peroxide pastes. However, the addition of water, apart from the water associated with water damp diaroyl peroxides, subsequent to the addition of paraffin oil was unexpectedly found to provide a further reduction in paste viscosity. This further decrease in diaroyl peroxide paste viscosity does not occur when substantially all of the water in the composition is blended with the diaroyl peroxide and other ingredients in a single step, which is the typical practice in the industry.

In some embodiments, the diaroyl peroxide paste includes a dispersant. In some embodiments, the dispersant is water. In further embodiments, the dispersant may be a polar compound or combination of polar compounds. Examples of suitable polar compounds include ethylene glycol, propylene glycol, glycerin, propylene carbonate, and hexylene glycol. In some embodiments, the diaroyl peroxide paste includes an amount of dispersant within the range of about 0.1% to about 40% by weight, within the range of about 5% to about 35% by weight, in within the range of about 12% to about 30% by weight, or within the range of about 16% to about 26% by weight.

In some embodiments, the diaroyl peroxide paste includes a preservative. Exemplary preservatives include tin-containing compounds, organophosphates, alkali phosphates, and benzylamides. In certain embodiments, the preservative is acetanilide. In some embodiments, the diaroyl peroxide paste includes an amount of preservative, by weight, within the range of about 0% to about 5%, within the range of about 0.1% to about 5%, within the range of about 0.1% to about 1%, within the range of about 0.1% to about 0.5%, or less than 1% or less than 0.5%.

Salt may be optionally added to the diaroyl peroxide paste. The addition of salt improves the water stability of the diaroyl peroxide paste, reducing the separation of water. Salt may be added at any point during the manufacturing process. In certain embodiments, salt is preferably added subsequent to the addition of paraffin oil as it is more easily blended into the paste once the viscosity has been decreased. In specific embodiments where the diaroyl peroxide paste includes water as a dispersant, the salt is a water soluble salt. In certain embodiments, the salt is sodium chloride. In other embodiments, other salts or mixtures of salts may be used. Exemplary salts include alkali carboxylic acids such as sodium benzoate, sodium oxalate, sodium acetate, potassium citrate, and combinations thereof. In some embodiments, the diaroyl peroxide paste includes an amount of salt within the range of about 0% to about 5% by weight. In further embodiments, the diaroyl peroxide paste includes an amount of salt within the range of about 0.1% to about 4% by weight. In further embodiments, the diaroyl peroxide paste includes an amount of salt within the range of about 0.5% to about 3% by weight. In certain embodiments, the diaroyl peroxide paste includes an amount of salt with the range of about 1% to about 2% by weight.

In some embodiments, the diaroyl peroxide composition includes a phase stabilizer to improve the stability of water in the dispersion. In certain embodiments, the phase stabilizer is modified or unmodified cellulose. Cellulose may be modified by acetate or butyrate or other compounds known in the art. Cellulose and modified cellulose act as phase stabilizers at amounts of less than about 1% by weight in the composition, and as thickening agents at concentrations of greater than about 1% by weight. In some embodiments, the diaroyl peroxide paste includes an amount of stabilizer, by weight, within the range of about 0% to about 5%, within the range of about 0.1% to about 5%, within the range of about 0.1% to about 1%, within the range of about 0.1% to about 0.5%, or less than 1% or less than 0.5%.

The diaroyl peroxide composition resulting from the process disclosed includes, by weight, greater than about 45% diaroyl peroxide, greater than about 50% diaroyl peroxide, greater than about 53% diaroyl peroxide, within the range of about 53% to about 56.5% diaroyl peroxide, or about 55% diaroyl peroxide. In some embodiments, the diaroyl peroxide composition has a freezing point below −20° C., below −30° C., or below −40° C. The diaroyl peroxide composition resulting from the process disclosed herein has a viscosity of less than about 10,000 cP, less than about 5,000 cP, less than about 3,000 cP, less than about 1,000 cP or less than about 500 cP. All viscosity values discussed herein are measured at or intended to be measured at 20° C.

Embodiments of diaroyl peroxide composition disclosed herein are suitable for use in securing a mine bolt, when combined with at least one of a carbonate, phosphate, sulfate or alkaline mineral. In some embodiments, the composition is combined with calcium carbonate, commonly known as limestone, or calcium sulfate. In some embodiments, the composition further includes a polymer resin and a thickening agent. In certain embodiments, the thickening agent may include cellulose or modified cellulose in the amount of greater than 1% by weight, and typically in the range of about 2% to 3% by weight. In further embodiments, thickening agents may include polyethylene glycol, polysaccharides, or lectin.

The following examples are provided to aid the understanding of the present invention, and it is understood that modification can be made in the procedures set forth without departing from the spirit of the invention.

### Example 1

In one embodiment, a dispersion in paste form comprises:

| Component | Percent Weight |
|---|---|
| Benzoyl Peroxide | 54-55% |
| Water | 20-22% |
| Paraffin Oil | 10-12% |
| Isodecyl Benzyl Ester | 6-8% |
| Surfactant | 0.1-3% |
| Sodium Chloride | 1-2% |
| Acetanilide | <0.5% |

In this embodiment, water damp BPO, isodecyl benzyl ester, acetanilide, and surfactant are combined in a planetary mixer. The components are blended for 10-20 minutes until mixture is a uniform doughy mixture. The mixing is stopped and the dough is allowed to remain still for at least 30 minutes. Paraffin oil is then added, and the composition is gently mixed. Mixing continues until the composition attains a uniform, flowable viscosity. This typically takes no longer than 10 minutes. The BPO concentration of the composition is typically determined at this point, as commercially available water damp BPO may be provided with variable water content. The amount of water needed to obtain the desired final BPO concentration is calculated. Sodium chloride and the balance of the water are then added to reach the desired BPO concentration. In this example, the resulting compound at 54.5% BPO by weight was then tested using the procedure described in ASTM-298, modified to use an autotitrator instead of manual titration. The procedure experimentally determined the compound to have a viscosity under 500 cP. The mixture was stored at 20° C. for 72 hours and water separation was visually observed to be less than 5% of total volume. The freezing point was measured to be below −20° C.

### Example 2

In one embodiment, a dispersion in paste form comprises:

| Component | Percent Weight |
|---|---|
| Benzoyl Peroxide | 54-55% |
| Water | 18-21% |
| Paraffin Oil | 14-16% |
| Isodecyl Benzyl Ester | 6-8% |
| Surfactant | 0.1-3% |

In this embodiment, water damp BPO, isodecyl benzyl ester, and surfactant are combined in a planetary mixer. The components are blended for 10-20 minutes until mixture is a uniform doughy mixture. The mixing is stopped and the dough is allowed to remain still for at least 30 minutes. Paraffin oil is then added, and the composition is gently mixed. Mixing continues until the composition attains a uniform, flowable viscosity. This typically takes no longer than 10 minutes. The BPO concentration of the composition is typically determined at this point, as commercially available water damp BPO can be provided with variable water content. The amount of water needed to obtain the desired final BPO concentration is calculated. The balance of the water is then added to reach the desired BPO concentration. In this example, the resulting compound at 55% BPO by weight was then tested using the procedure described in ASTM-298, modified to use an autotitrator instead of manual titration. The procedure experimentally determined the compound to have a viscosity under 1,500 cP. The mixture was stored at 20° C. for 72 hours and water separation was visually observed to be less than 10% of total volume. The freezing point was measured to be below −30° C.

### Example 3

In one embodiment, a dispersion in paste form comprises:

| Component | Percent Weight |
|---|---|
| Benzoyl Peroxide | 54-55% |
| Water | 16-18% |
| Paraffin Oil | 18-20% |
| Isodecyl Benzyl Ester | 6-8% |
| Surfactant | 0.1%-3% |
| Acetanilide | <0.5% |
| Modified Cellulose | <0.3% |

In this embodiment, water damp BPO, isodecyl benzyl ester, modified cellulose, and surfactant are combined in a planetary mixer. The components are blended for 10-20 minutes until mixture is a uniform doughy mixture. The mixing is stopped and the dough is allowed to remain still for at least 30 minutes. Paraffin oil is then added, and the composition is gently mixed. Mixing continues until the composition attains a uniform, flowable viscosity. This typically takes no longer than 10 minutes. The BPO concentration of the composition is typically determined at this point, as commercially available water damp BPO can be provided with variable water content. The amount of water needed to obtain the desired final BPO concentration is calculated. The balance of the water is then added to reach the desired BPO concentration. In this example, the resulting compound at 55% BPO by weight was then tested using the procedure described in ASTM-298, modified to use an autotitrator instead of manual titration. The procedure experimentally determined the compound to have a viscosity under 2500 cP. The mixture was stored at 20° C. for 72 hours and water separation was visually observed to be less than 5% of total volume. The freezing point was measured to be below −40° C.

### Example 4

In one embodiment, a dispersion in paste form comprises:

| Component | Percent Weight |
|---|---|
| Benzoyl Peroxide | 54-55% |
| Water | 22-26% |
| Paraffin Oil (solvent isolated) | 8-10% |
| Isodecyl Benzyl Ester | 6-8% |
| Surfactant | 0.1-3% |
| Sodium Chloride | 1-2% |
| Acetanilide | <0.5% |

In this embodiment, the water damp BPO, isodecyl benzyl ester and surfactant were combined in a planetary mixer. The components were blended for 10-20 minutes until mixture was a uniform doughy mixture. The mixing is stopped and the dough is allowed to remain still for a period time of at least 30 minutes. Solvent isolated paraffin oil with a dark yellow color is then added, and mixing is gently applied. The mixture was blended until a uniform, flowable viscosity was attained. This typically takes no longer than about 10 minutes. Sodium chloride is then added. The resulting paste was of a yellowish color with a creamy appearance and a mild ester aroma. The BPO concentration of the mixture is determined. The amount of extra dispersant was calculated to achieve the desired final BPO concentration, and the balance of the water added. In this example, the resulting compound at 54.5% BPO by weight was then tested using the procedure described in ASTM-298, modified to use an autotitrator instead of manual titration. The procedure experimentally determined the compound to have a viscosity under 300 cP. The mixture was stored at 20° C. for 72 hours and water separation was visually observed to be less than 5% of total volume. The freezing point was measured to be below −20° C.

### Example 5

In one embodiment, a dispersion in paste form comprises:

| Component | Percent Weight |
|---|---|
| Benzoyl Peroxide | 54-55% |
| Water | 22-26% |
| Paraffin Oil (solvent isolated) | 10-12% |
| Isodecyl Benzyl Ester | 6-8% |
| Surfactant | 0.1-3% |
| Acetanilide | <0.5% |

In this embodiment, the water damp BPO, isodecyl benzyl ester and surfactant were combined in a planetary mixer. The components were blended for 10-20 minutes until mixture was a uniform doughy mixture. The mixing was stopped and the dough was allowed to remain still for a period time of at least 30 minutes. Solvent isolated paraffin oil with a dark yellow color was then added, and mixing was gently applied. The mixture was blended until a uniform, flowable viscosity was attained. This takes usually no longer than 10 minutes. The resulting paste was of a yellowish color with a creamy appearance and a mild ester aroma. The BPO concentration of the mixture was determined. The amount of extra dispersant was calculated to achieve the desired final assay, and the balance of the dispersant added. In this example, the resulting compound at 55.0% BPO by weight was then tested using the procedure described in ASTM-298, modified to use an autotitrator instead of manual titration. The procedure experimentally determined the compound to have a viscosity under 500 cP. The mixture was stored at 20° C. for 72 hours and water separation was visually observed to be less than 10% of total volume. The paste could be easily remixed by hand to make a uniform mixture that could be used at the moment. If the paste was allowed to stand for another 72 hours, water would again separate and observed to be less than 10% of total volume. The freezing point was measured to be below −10° C.

### Example 6

In one embodiment, a dispersion in paste form comprises:

| Component | Percent Weight |
|---|---|
| Benzoyl Peroxide | 54-55% |
| Water | 18-21% |
| White Paraffin Oil | 14-16% |
| Isodecyl Benzyl Ester | 6-8% |
| Surfactant | 0.1-3% |
| Acetanilide | <0.5% |

In this embodiment, the water damp BPO, isodecyl benzyl ester and surfactant were combined in a planetary mixer. The components were blended for 10-20 minutes until mixture was a uniform doughy mixture. The mixing was stopped and the dough is allowed to remain still for a period time of at least 30 minutes. White paraffin oil was then added, and mixing was gently applied. The mixture was blended until a uniform, flowable viscosity was attained. This takes usually no longer than 10 minutes. The BPO concentration of the mixture was determined. The amount of extra water was calculated to achieve the desired final BPO concentration, and the balance of the water added. In this example, the resulting compound at 55% BPO by weight was then tested using the procedure described in ASTM-298, modified to use an autotitrator instead of manual titration. The procedure experimentally determined the compound to have a viscosity under 1,500 cP. The resulting paste was of white color with a uniform creamy appearance and a mild ester aroma. The mixture was stored at 20° C. for 72 hours and water separation was visually observed to be less than 10% of total volume. The freezing point was measured to be below −30° C.

### Example 7

In one embodiment, a dispersion in paste form comprises:

| Component | Percent Weight |
|---|---|
| Benzoyl Peroxide | 54-55% |
| Water | 16-18% |
| White Paraffin Oil | 18-20% |
| Isodecyl Benzyl Ester | 6-8% |
| Surfactant | 0.1-3% |
| Acetanilide | <0.5% |
| Modified Cellulose | <0.3% |

In this embodiment, the water damp BPO, isodecyl benzyl ester and surfactant were combined in a planetary mixer. The components were blended for 10-20 minutes until mixture is a uniform doughy mixture. The mixing was stopped and the dough was allowed to remain still for a period time of at least 30 minutes. White paraffin oil was then added, and mixing was gently applied. The mixture was blended until a uniform, flowable viscosity was attained. This takes usually no longer than 10 minutes. The BPO concentration of the mixture was determined. The amount of extra water was calculated to achieve the desired final BPO concentration, and the balance of the water added. In this example, the resulting compound at 55% BPO by weight was then tested using the procedure described in ASTM-298, modified to use an autotitrator instead of manual titration. The procedure experimentally determined the compound to have a viscosity under 2,500 cP. The resulting paste was of white color with a uniform creamy appearance. The mixture was stored at 20° C. for 72 hours and water separation was visually observed to be less than 5% of total volume. The freezing point was measured to below −40° C.

### Example 8

A sample of this paste generated in Example 7 was mixed at a 5% weight concentration with limestone to generate a composition suitable for use as a mine bolt hardener. A corresponding control sample of a commercially available 55% BPO high viscosity paste was also made into 5% weight mixture with limestone to generate a composition suitable for use as a mine bolt hardener. Both mixtures were placed in a glass scintillation vial with a cap tightly fastened. These vials were put into a forced air convention oven set at 50° C. for two weeks. The BPO content of each mixture was measured using procedure described in ASTM-298. The mixture made using the paste generated in Example 7 retained nearly 85% of the original BPO content where the control sample retained only about 70% of the original BPO content.

### Example 9

In one embodiment, a dispersion in paste form comprises:

| Component | Percent Weight |
|---|---|
| Benzoyl Peroxide | 54-55% |
| Water | 22-26% |
| Naptha Solvent | 8-10% |
| Isodecyl Benzyl Ester | 6-8% |
| Surfactant | 0.1-3% |
| Sodium Chloride | 1-2% |
| Acetanilide | <0.5% |

In this embodiment, the water damp BPO, isodecyl benzyl ester, and surfactant were combined in a planetary mixer. The components were blended for 10-20 minutes until mixture was a uniform doughy mixture. The mixing was stopped and the dough was allowed to remain still for a period time of at least 30 minutes. Naptha solvent was then added, and mixing was gently applied. The mixture was blended until a uniform, flowable viscosity was attained. This takes usually no longer than 10 minutes. Finally, sodium chloride was added. The resulting paste was of a white color with a uniform but slightly grainy appearance and a strong naptha odor. The BPO concentration of the mixture was determined. The amount of extra dispersant is calculated to achieve the desired final concentration, and the balance of the dispersant added. In this example, the resulting compound at 54.7% BPO by weight was then tested using the procedure described in ASTM-298, modified to use an autotitrator instead of manual titration. The procedure experimentally determined the compound to have a viscosity under 500 cP. The mixture was stored at 20° C. for 72 hours and the amount of separation of clear liquids was visually observed to be less than 20% of total volume.

### Example 10

In one embodiment, a dispersion in paste form comprises:

| Component | Percent Weight |
|---|---|
| Benzoyl Peroxide | 54-55% |
| Water | 16-18% |
| White Paraffin Oil | 18-20% |
| Isodecyl Benzyl Ester | 6-8% |
| Surfactant | 0.1-3% |
| Acetanilide | <0.5% |
| Modified Cellulose | <0.3% |
| Sodium Chloride | 1-2% |

In this embodiment, the water damp BPO, isodecyl benzyl ester, and surfactant were combined in a planetary mixer. The components were blended for 10-20 minutes until mixture was a uniform doughy mixture. The mixing was stopped and the dough was allowed to remain still for a period time of at least 30 minutes. White paraffin oil was then added, and mixing was gently applied. The mixture was blended until a uniform, flowable viscosity was attained. This takes usually no longer than 10 minutes. Finally, sodium chloride was added. The resulting paste was of a white color with a creamy but slightly grainy appearance and a strong kerosene odor. The concentration of the mixture was determined. The amount of extra dispersant is calculated to achieve the desired final concentration, and the balance of the dispersant added. In this example, the resulting compound at 54.9% BPO by weight was then tested using the procedure described in ASTM-298, modified to use an autotitrator instead of manual titration. The procedure experimentally determined the compound to have a viscosity under 500 cP. The mixture was stored at 20° C. for 72 hours and the amount of separation of clear liquids was visually observed to be less than 15% of total volume.

### Example 11

In one embodiment, a dispersion in paste form comprises:

| Component | Percent Weight |
|---|---|
| Benzoyl Peroxide | 54-55% |
| Water | 16-18% |
| White Paraffin Oil | 18-20% |
| Isodecyl Benzyl Ester | 6-8% |
| Surfactant | 0.1-3% |
| Acetanilide | <0.5% |
| Modified Cellulose | <0.3% |

In this embodiment, the BPO, water, isodecyl benzyl ester, surfactant, acetanilide, paraffin oil and modified cellulose were combined in a planetary mixer. The components were blended for 10-20 minutes under vacuum until mixture was uniform in appearance. The BPO concentration of the mixture was determined. The amount of extra dispersant was calculated to achieve the desired final concentration, and the balance of the dispersant added. In this example, the resulting compound at 55% BPO by weight was then tested using the procedure described in ASTM-298, modified to use an autotitrator instead of manual titration. The procedure experimentally determined the compound to have a viscosity under 35,000 cP. The resulting paste was of white color with a uniform creamy appearance. The mixture was stored at 20° C. for 72 hours and water separation was visually observed to be less than 5% of total volume. The freezing point was measured to below −40° C. The mixture was allowed to stand for 4 weeks and the viscosity of the paste was measured to be 25,000 cP.

### Example 12

In one embodiment, a stabilized dispersion in paste form comprises:

| Component | Percent Weight |
|---|---|
| Benzoyl Peroxide | 54-55% |
| Water | 16-18% |
| White Paraffin Oil | 18-20% |
| Isodecyl Benzyl Ester | 6-8% |
| Surfactant | 0.1-3% |
| Acetanilide | <0.5% |
| Modified Cellulose | <0.3% |

In this embodiment, the BPO, isodecyl benzyl ester, and surfactant were combined in a planetary mixer. The components were blended for 10-20 minutes until mixture is a uniform doughy mixture. The mixing was stopped and the dough was allowed to remain still for a period time of at least 30 minutes. White paraffin oil was then added, and mixing was vigorously applied for 15 minutes. The BPO concentration of the mixture was determined. The amount of extra dispersant was calculated to achieve the desired final BPO concentration. In this example, the resulting compound at 55% BPO by weight was then tested using the procedure described in ASTM-298, modified to use an autotitrator instead of manual titration. The procedure experimentally determined the compound to have a viscosity under 30,000 cP. The resulting paste was of white color with a uniform creamy appearance with a mild ester aroma. The mixture was stored at 20° C. for 72 hours and water separation was visually observed to be less than 5% of total volume. The freezing point was measured to below −40° C. The mixture was allowed to stand for 4 weeks and the viscosity of the paste was measured to be 3,500 cP. As compared to the composition from Example 11, adding the paraffin oil after a waiting period, rather than in the initial mixing, resulted in a significantly lower viscosity after being allowed to stand for 4 weeks.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1. A method of making a diaroyl peroxide paste comprising:
blending a diaroyl peroxide, at least one surfactant, and at least one diluent;
waiting a predetermined period of time; and
adding paraffin oil;
wherein the paste is at least about 45% by weight diaroyl peroxide and has a viscosity of less than about 10,000 cP.

2. The method of claim 1, wherein the paste is not agitated during the waiting.

3. The method of claim 1, wherein the predetermined period of time is at least 5 minutes.

4. The method of claim 3, wherein the predetermined period of time is at least 30 minutes.

5. The method of claim 1, further comprising adding a dispersant.

6. The method of claim 5, wherein the dispersant is water.

7. The method of claim 6, wherein the water is added subsequent to the paraffin oil.

8. The method of claim 6, further comprising adding a water soluble salt.

9. The method of claim 1, further comprising adding salt.

10. The method of claim 9, wherein the salt is added subsequent to the addition of paraffin oil.

11. The method of claim 9, wherein the salt is one of sodium chloride, an alkali carboxylic acid, or mixture thereof.

12. The method of claim 1, wherein the diaroyl peroxide is dibenzoyl peroxide.

13. The method of claim 1, wherein the at least one diluent is one of a phthalate, benzoate and dibenzoate, phosphate, citrate, or adipate, or mixtures thereof.

14. The method of claim 13, wherein the at least one diluent is a benzyl ester or mixture of benzyl esters.

15. The method of claim 1, wherein the paste is at least about 50% by weight diaroyl peroxide, and wherein the predetermined period of time is at least 15 minutes.

16. The method of claim 1, wherein the paste is at least about 53% by weight diaroyl peroxide and has a viscosity of less than about 5,000 cP.

17. A diaroyl peroxide composition comprising:
at least about 45% by weight diaroyl peroxide;
between about 2% and about 15% by weight diluent;
between about 0.1% and about 10% by weight surfactant; and
between about 1% and about 30% by weight paraffin oil;
wherein the composition has a viscosity of less than about 10,000 cP and a freezing point of less than about −20° C.

18. The composition of claim 17, wherein the composition includes at least about 50% by weight diaroyl peroxide, between about 4% and about 10% by weight diluent, between about 0.5% and about 7% by weight surfactant, and between about 5% and about 25% by weight paraffin oil; and
wherein the composition has a viscosity of less than about 5,000 cP.

19. The composition of claim 17, further comprising between about 5% and about 35% by weight dispersant.

20. The composition of claim 19, wherein the dispersant is water.

21. The composition of claim 17, wherein the composition includes at least about 50% by weight diaroyl peroxide and has a viscosity of less than about 5,000 cP.

22. The composition of claim 21, having a viscosity of less than about 1,000 cP.

23. A diaroyl peroxide composition comprising:
at least about 50% by weight diaroyl peroxide;
between about 4% and about 10% by weight diluent;
between about 6% and about 24% by weight paraffin oil; and
between about 12% and about 30% by weight dispersant;
wherein the composition has a viscosity of less than about 10,000 cP and a freezing point of less than about −20° C.

24. The composition of claim 23, further comprising between about 0.1% and about 5% by weight of a stabilizer or a preservative.

25. The composition of claim 23, wherein the dispersant is water.

26. The composition of claim 23, further comprising at least one of a carbonate, phosphate, sulfate or alkaline mineral.

27. The composition of claim 23, further comprising calcium carbonate or calcium sulfate.

28. The composition of claim 23, further comprising a polymer resin and a thickening agent.

29. A diaroyl peroxide composition comprising:
at least about 50% by weight diaroyl peroxide;
between about 4% and about 10% by weight diluent;
between about 0.5% and about 7% by weight surfactant; and
between about 5% and about 25% by weight non-polar solvent with an octanol partition log p of at least about 5;
wherein the composition has a viscosity of less than about 5,000 cP.

30. The composition of claim 28, wherein the non-polar solvent is one of paraffin oil, naptha or kerosene.
